(19) United States
(12) Patent Application Publication
(10) Pub. No.: US 2025/0249058 A1
(43) Pub. Date: Aug. 7, 2025

(54) MELASTOMA DODECANDRUM PROANTHOCYANIDIN ANALOG AND EXTRACTION METHOD THEREOF

(71) Applicants: Zhejiang University, Hangzhou City (CN); Ningbo Innovation Center, Zhejiang University, Ningbo City (CN)

(72) Inventors: Wei CHEN, Hangzhou City (CN); Yang XU, Hangzhou City (CN); Lianghua XIE, Hangzhou City (CN); Dongyuan HOU, Hangzhou City (CN)

(21) Appl. No.: 18/815,070

(22) Filed: Aug. 26, 2024

(30) Foreign Application Priority Data
Feb. 2, 2024 (CN) 202410158054.4

(51) Int. Cl. A61K 36/185; A61K 31/353; A61P 3/08; B01D 11/02; B01D 11/04; B01D 15/42

(52) U.S. Cl. CPC A61K 36/185; A61K 31/353; A61P 3/08; B01D 11/0265; B01D 11/0288; B01D 11/0492; B01D 15/426

(57) ABSTRACT

Provided are a Melastoma dodecandrum proanthocyanidin analog and an extraction method thereof. In the method, Melastoma dodecandrum fruit is subjected to alcohol extraction to obtain a crude extract of Melastoma dodecandrum Lour. The crude extract of the Melastoma dodecandrum Lour. is subjected to extraction with ethyl acetate to obtain a Melastoma dodecandrum extract, and the Melastoma dodecandrum extract is subjected to preparative liquid chromatography purification to obtain the Melastoma dodecandrum proanthocyanidin analog.

### CROSS REFERENCE TO RELATED APPLICATION

This patent application claims the benefit and priority of Chinese Patent Application No. 2024101580544 filed with the China National Intellectual Property Administration on Feb. 2, 2024, the disclosure of which is incorporated by reference herein in its entirety as part of the present application.

### TECHNICAL FIELD

The present disclosure relates to the technical field of plant extraction, in particular to a Melastoma dodecandrum proanthocyanidin analog and an extraction method thereof.

### BACKGROUND

Diabetes is one of the common chronic diseases worldwide. Diabetics are usually accompanied by hyperglycemia. Long-term hyperglycemia can cause damage to tissues and organs such as nerves, heart, blood vessels and kidneys, and cause a plurality of acute and chronic complications. According to the IDF Diabetes Atlas Report, the number of the diabetics increased from 108 million in 1980 to 537 million in 2021. Prevalence rates in low- and middle-income countries are rising faster than those in high-income countries. The diabetes is prone to a plurality of complications, and treatment attention needs to be improved urgently. Therefore, research on the prevention and treatment of the diabetes is of great significance.

Dipeptidyl peptidase-4 (DPP4) is a serine protease widely present in tissues such as kidneys, gastrointestinal tract, and lymph nodes. DPP-4 inhibitors, namely dipeptidyl peptidase-4 inhibitors, are a class of drugs for the treatment of type 2 diabetes. Pharmacologically, these drugs inhibit the inactivation of glucagon-like peptide-1 (GLP-1) and glucose-dependent insulinotropic polypeptide (GIP), increase the levels of endogenous GLP-1 and GIP, promote the release of insulin from islet β cells, and suppress the secretion of glucagon by islet α cells, thereby increasing insulin levels and reducing blood glucose. These drugs are not susceptible to hypoglycemia and weight gain. Clinical studies have shown that DPP-4 inhibitors can reduce HbA1c by 0.4-0.9%. At present, the DPP-4 inhibitors marketed in China mainly include sitagliptin, alogliptin, linagliptin, vildagliptin and saxagliptin. However, these drugs are chemically synthetic drugs, with a plurality of side effects, such as triggering hypersensitivity, elevated liver enzymes, upper respiratory tract infections, angioedema, rashes, pancreatitis, etc. Therefore, exploring new safe and effective DPP4 inhibitors from natural edible plants is currently a research hotspot in the field of food and medicine.

Melastoma dodecandrum Lour. is a plant in the family Melastomastaceae. The Melastoma dodecandrum Lour is a commonly used folk medicine of the She people and has been included in the 2015 edition of the Traditional Chinese Medicine Processing Standards of Zhejiang Province. The Melastoma dodecandrum Lour. is native to southern China, mainly distributed in the hillside grasslands and forests below 1300 m above sea level in provinces such as Jiangxi, Fujian, and Zhejiang south of the Yangtze River. The Melastoma dodecandrum Lour. prefers acidic soil, has very strong vitality, and is characteristic of cold resistance, drought resistance, and rapid growth. The Melastoma dodecandrum fruit is rich in nutrients such as polyphenols. The Melastoma dodecandrum fruit is not only directly edible, but also for medicinal use. However, so far, the utilization of Melastoma dodecandrum Lour. is still based on the development of wild resources. There is no effective artificial propagation and cultivation on a large scale. Due to the limited resources, there are few studies on the phytochemical composition and functional activity of Melastoma dodecandrum Lour.

### SUMMARY

In view of this, the present disclosure is intended to provide a Melastoma dodecandrum proanthocyanidin analog and an extraction method thereof. The Melastoma dodecandrum proanthocyanidin analog extracted in the present disclosure has good inhibitory activity against DPP4 with few side effects.

To achieve the above objects, the present disclosure provides the following technical solutions.

The present disclosure provides a method for extracting a Melastoma dodecandrum proanthocyanidin analog, including
subjecting Melastoma dodecandrum fruit to alcohol extraction to obtain a crude extract of Melastoma dodecandrum Lour.

The crude extract of the Melastoma dodecandrum Lour is then subject to extraction with ethyl acetate to obtain a Melastoma dodecandrum extract.

The Melastoma dodecandrum extract is subject to preparative liquid chromatography purification to obtain the Melastoma dodecandrum proanthocyanidin analog.

The preparative liquid chromatography purification is conducted under parameters including a mobile phase A is pure acetonitrile, and a mobile phase B is an aqueous formic acid solution with a formic acid concentration of 1% to 2% by volume.

Gradient elution is programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A.

A flow rate is 8-10 mL/min, a column temperature is 30° C., and a detection wavelength is 280 nm; and
a fraction at a retention time of 26.5-27.2 min is collected based on a liquid chromatogram.

In some embodiments, a reagent for the alcohol extraction is an ethanol solution; the ethanol solution has a volume concentration of 50% to 80%; the ethanol solution further includes a 0.1% by volume of acid; and the acid is one selected from the group consisting of hydrochloric acid, formic acid, and acetic acid.

In some embodiments, the alcohol extraction is conducted at a feed-to-liquid ratio of 1 g:4 mL to 1 g:20 mL; the alcohol extraction is performed by one selected from the group consisting of magnetic stirring extraction in a water bath and ultrasonic extraction; and the alcohol extraction is conducted for 60-240 min.

In some embodiments, after the alcohol extraction, the method further includes: subjecting a resulting extraction material to vacuum filtration to obtain a filtrate; and subjecting the filtrate to evaporation to remove the reagent for the alcohol extraction from the filtrate, where the evaporation is rotary vacuum evaporation, and the rotary vacuum evaporation is conducted at a temperature of 40° C. to 60° C.

In some embodiments, a volume ratio of the ethyl acetate to the crude extract of the Melastoma dodecandrum Lour. is in a range of 0.5:1 to 3:1, and the extraction. is conducted 2-6 times.

In some embodiments, after the extraction, an ethyl acetate phase is collected; the ethyl acetate phase is subjected to evaporation to remove the ethyl acetate from the ethyl acetate phase; the evaporation is rotary vacuum evaporation, and the rotary vacuum evaporation is conducted at a temperature of 40° C. to 50° C.

In some embodiments, the preparative liquid chromatography purification is conducted twice; and a second preparative liquid chromatography purification is conducted under parameters including: the mobile phase A is the pure acetonitrile, and the mobile phase B is the aqueous formic acid solution with the formic acid concentration of 1% to 2% by volume; the gradient elution is programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A; the flow rate is 8-10 mL/min, the column temperature is 30° C., and the detection wavelength is 280 nm; and the fraction at a retention time of 26.8-27.2 min is collected based on the liquid chromatogram.

In some embodiments, after the fraction is collected, the method further includes subjecting the fraction to vacuum evaporation and freeze-drying in sequence.

The present disclosure further provides a Melastoma dodecandrum proanthocyanidin analog prepared by the method described in the above technical solution, where an active pharmaceutical ingredient of the Melastoma dodecandrum proanthocyanidin analog is a proanthocyanidin trimer.

The present disclosure further provides use of the Melastoma dodecandrum proanthocyanidin analog described in the above technical solution in preparation of hypoglycemic drugs.

The present disclosure provides a method for extracting a Melastoma dodecandrum proanthocyanidin analog, including the following steps: subjecting Melastoma dodecandrum fruit to alcohol extraction to obtain a crude extract of Melastoma dodecandrum Lour.; subjecting the crude extract of the Melastoma dodecandrum Lour. to extraction with ethyl acetate to obtain a Melastoma dodecandrum extract; and subjecting the Melastoma dodecandrum extract to preparative liquid chromatography purification to obtain the Melastoma dodecandrum proanthocyanidin analog; where the preparative liquid chromatography purification is conducted under parameters including: a mobile phase A is pure acetonitrile, and a mobile phase B is an aqueous formic acid solution with a formic acid concentration of 1% to 2% by volume; gradient elution is programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A; a flow rate is 8-10 mL/min, a column temperature is 30° C., and a detection wavelength is 280 nm; and a fraction at a retention time of 26.5-27.2 min is collected based on the liquid chromatogram. The Melastoma dodecandrum proanthocyanidin analog extracted in the present disclosure has significant inhibitory activity against DPP4, and the Melastoma dodecandrum proanthocyanidin analog is extracted from natural plants with few side effects.

The present disclosure further provides a Melastoma dodecandrum proanthocyanidin analog obtained by the method according to the above technical solution, where an active pharmaceutical ingredient of the Melastoma dodecandrum proanthocyanidin analog is a proanthocyanidin trimer. Animal experiments have shown that the Melastoma dodecandrum proanthocyanidin analog could significantly reduce mouse postprandial blood glucose (PBG), showing excellent hypoglycemic activity in vivo. In vitro experiments have shown that the Melastoma dodecandrum proanthocyanidin analog could reduce PBG in Institute of Cancer Research (ICR) mice by inhibiting dipeptidyl peptidase-4 (DPP4) activity, showing excellent hypoglycemic activity. Therefore, the Melastoma dodecandrum proanthocyanidin analog can be used as hypoglycemic functional foods or hypoglycemic drugs for the prevention and treatment of diseases related to abnormal glucose metabolism. The present disclosure provides a new idea for the comprehensive development and utilization of Melastoma dodecandrum Lour. resources in China.

### BRIEF DESCRIPTION OF THE DRAWINGS

FIG. 1 shows a high performance liquid chromatography (HPLC) chromatogram of the Melastoma dodecandrum extract in Example 1;

FIG. 2 shows an HPLC chromatogram of the first purified Melastoma dodecandrum proanthocyanidin analog in Example 2;

FIG. 3 shows an HPLC chromatogram of the second purified Melastoma dodecandrum proanthocyanidin analog in Example 2;

FIG. 4 is a diagram showing the inhibitory effect of Melastoma dodecandrum proanthocyanidin analog in Example 3 on DPP4; and

FIGS. 5A-5B are diagrams showing the hypoglycemic effect of Melastoma dodecandrum proanthocyanidin analog in Example 4 in animals.

### DETAILED DESCRIPTION OF THE EMBODIMENTS

The present disclosure provides a method for extracting a Melastoma dodecandrum proanthocyanidin analog, including the following steps:
subjecting Melastoma dodecandrum fruit to alcohol extraction to obtain a crude extract of Melastoma dodecandrum Lour.;
subjecting the crude extract of the Melastoma dodecandrum Lour to extraction with ethyl acetate to obtain a Melastoma dodecandrum extract; and
subjecting the Melastoma dodecandrum extract to preparative liquid chromatography purification to obtain the Melastoma dodecandrum proanthocyanidin analog, where
the preparative liquid chromatography purification is conducted under parameters including:
a mobile phase A is pure acetonitrile, and a mobile phase B is an aqueous formic acid solution with a formic acid concentration of 1% to 2% by volume;
gradient elution is programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A;
a flow rate is 8-10 mL/min, a column temperature is 30° C., and a detection wavelength is 280 nm; and
a fraction at a retention time of 26.5-27.2 min is collected based on a liquid chromatogram.

In some embodiments of the present disclosure, unless otherwise specified, all raw materials used in the present disclosure are commercially available products.

In the present disclosure, Melastoma dodecandrum fruit is subjected to alcohol extraction to obtain a crude extract of Melastoma dodecandrum Lour.

In some embodiments of the present disclosure, the Melastoma dodecandrum fruit is fresh Melastoma dodecandrum fruit. In some embodiments of the present disclosure, the Melastoma dodecandrum fruit is subjected to cleaning before the alcohol extraction.

In some embodiments of the present disclosure, a reagent for the alcohol extraction is an ethanol solution. In some embodiments of the present disclosure, the ethanol solution has a volume concentration of 50% to 80%, and preferably 60% to 70%. In some embodiments of the present disclosure, the ethanol solution further includes a 0.1% by volume of acid; and the acid is one selected from the group consisting of hydrochloric acid, formic acid, and acetic acid.

In some embodiments of the present disclosure, the alcohol extraction is conducted at a feed-to-liquid ratio of 1 g:4 mL to 1 g:20 mL, and preferably 1 g:8 mL to 1 g:15 mL, namely a dosage ratio of the Melastoma dodecandrum fruit to the reagent for the alcohol extraction.

In some embodiments of the present disclosure, the alcohol extraction is performed by one selected from the group consisting of magnetic stirring extraction in a water bath and ultrasonic extraction, and preferably the ultrasonic extraction. In some embodiments of the present disclosure, the magnetic stirring extraction in the water bath is conducted at a rotational speed of 800-1600 rpm. In some embodiments of the present disclosure, the ultrasonic extraction is conducted at a power of 50-100 W.

In some embodiments of the present disclosure, the alcohol extraction is conducted at room temperature, namely neither additional heating nor cooling is required; the alcohol extraction is conducted for 60-240 min.

In some embodiments of the present disclosure, after the alcohol extraction, the method further includes: subjecting a resulting extraction material to vacuum filtration to obtain a filtrate; and subjecting the filtrate to evaporation to remove the reagent for the alcohol extraction from the filtrate. In some embodiments of the present disclosure, the evaporation is rotary vacuum evaporation; the rotary vacuum evaporation is conducted at a temperature of 40° C. to 60° C.

In the present disclosure, after the crude extract of Melastoma dodecandrum Lour is obtained, the crude extract of the Melastoma dodecandrum Lour. is subjected to extraction with ethyl acetate to obtain a Melastoma dodecandrum extract.

In some embodiments of the present disclosure, a volume ratio of the ethyl acetate to the crude extract of the Melastoma dodecandrum Lour is in a range of 0.5:1 to 3:1, and preferably 1:1. In some embodiments of the present disclosure, the extraction is conducted 2-6 times. In the present disclosure, after each the extraction, an ethyl acetate phase is collected.

In some embodiments of the present disclosure, after the extraction, the ethyl acetate phase is collected; the ethyl acetate phase is subjected to evaporation to remove the ethyl acetate from the ethyl acetate phase. In some embodiments of the present disclosure, the evaporation is rotary vacuum evaporation; the rotary vacuum evaporation is conducted at a temperature of 40° C. to 50° C.

In the present disclosure, after the Melastoma dodecandrum extract is obtained, the Melastoma dodecandrum extract is subjected to preparative liquid chromatography purification to obtain the Melastoma dodecandrum proanthocyanidin analog.

In the present disclosure, the preparative liquid chromatography purification is conducted under parameters including:
a mobile phase A is pure acetonitrile, and a mobile phase B is an aqueous formic acid solution with a formic acid concentration of 1% to 2% by volume;
gradient elution is programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A;
a flow rate is 8-10 mL/min, a column temperature is 30° C., and a detection wavelength is 280 nm; and
a fraction at a retention time of 26.5-27.2 min is collected based on a liquid chromatogram.

In some embodiments of the present disclosure, before the Melastoma dodecandrum extract is subjected to the preparative liquid chromatography, the method further includes the step of preparing the Melastoma dodecandrum extract into a Melastoma dodecandrum extract solution, where the Melastoma dodecandrum extract solution has a concentration of 10 mg/mL, and a solvent for the Melastoma dodecandrum extract solution is dimethyl sulfoxide (DMSO).

In some embodiments of the present disclosure, the preparative liquid chromatography purification is conducted twice. In the present disclosure, parameters for the second preparative liquid chromatography purification are consistent with those of the first preparative liquid chromatography purification, and the fraction at a retention time of 26.8-27.2 min is collected based on the liquid chromatogram.

In the present disclosure, after the fraction is collected, vacuum evaporation and freeze-drying in sequence are further conducted in sequence.

The present disclosure further provides a Melastoma dodecandrum proanthocyanidin analog obtained by the method described in the above technical solution, where an active pharmaceutical ingredient of the Melastoma dodecandrum proanthocyanidin analog is a proanthocyanidin trimer. The Melastoma dodecandrum proanthocyanidin analog in the present disclosure is extracted from plants with fewer side effects compared with chemosynthetic DPP4.

The present disclosure further provides use of the Melastoma dodecandrum proanthocyanidin analog described in the above technical solution in preparation of hypoglycemic drugs.

The present disclosure does not impose specific limitations on the use of the Melastoma dodecandrum proanthocyanidin analog, as long as operations well known to those skilled in the art may be adopted.

The Melastoma dodecandrum proanthocyanidin analog and an extraction method thereof provided by the present disclosure will be described in detail below with reference to examples, but they should not be understood as limiting the scope of the present disclosure.

### Example 1 Preparation of a Melastoma dodecandrum Extract

(1) A total of 1000 g of fresh Melastoma dodecandrum fruit was washed, and mixed with ethanol solution with a volume concentration of 70% (containing 0.1% of hydrochloric acid in the ethanol solution) at a feed-to-liquid ratio of 1 g:8 mL. A resulting system was subjected to beating and then fully mixing, and a resulting mixture was subjected to ultrasonic extraction at 100 W for 90 min. After ultrasonic extraction, a resulting system was subjected to vacuum filtration to remove ethanol under rotary vacuum evaporation at 60° C. to obtain a crude extract of Melastoma dodecandrum Lour.
(2) The crude extract of the Melastoma dodecandrum Lour. was mixed with ethyl acetate at a volume ratio of 1:1 and let stand for layering. An ethyl acetate phase was collected. The extraction was repeated twice, and the ethyl acetate phases were collected and combined. The ethyl acetate phase mixture was subjected to rotary vacuum evaporation at a temperature of 40-50° C. to remove the ethyl acetate. A resulting system was subjected to freeze-drying to obtain the Melastoma dodecandrum extract. It was calculated that 5.6 g of the Melastoma dodecandrum extract could be obtained from 1000 g of the fresh Melastoma dodecandrum fruit after the above purification steps.

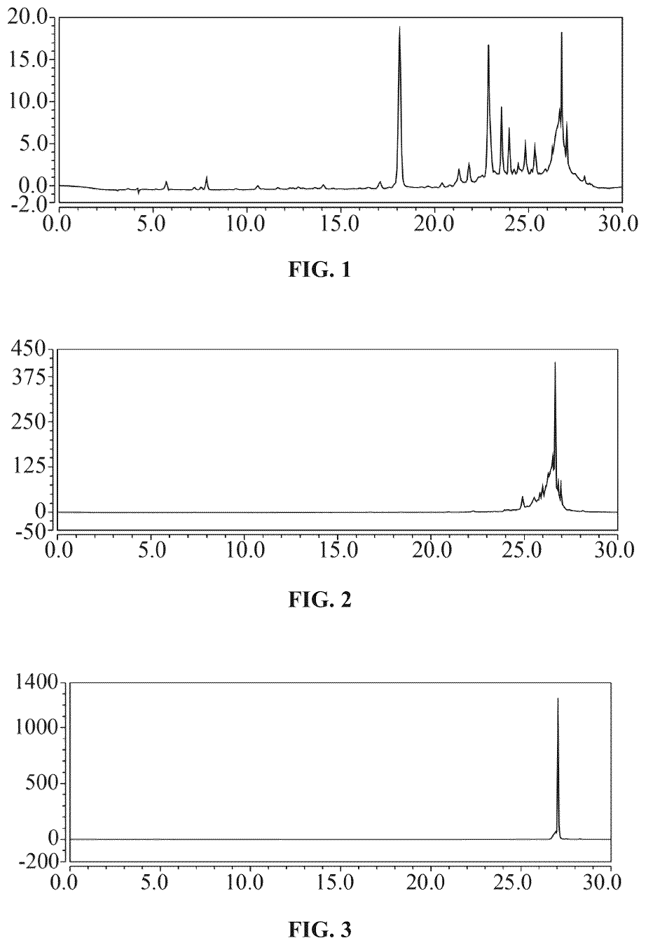

FIG. 1 shows an HPLC chromatogram of the resulting Melastoma dodecandrum extract. It can be seen from FIG. 1 that components are complex in the Melastoma dodecandrum extract.

### Example 2 Preparation of Melastoma dodecandrum Proanthocyanidin Analog

The Melastoma dodecandrum extract in Example 1 was prepared into a 10 mg/mL Melastoma dodecandrum extract solution with DMSO. The Melastoma dodecandrum extract solution was subjected to preparative liquid chromatography purification, which was conducted under the following parameters:
a mobile phase A was pure acetonitrile, and a mobile phase B was an aqueous formic acid solution with a formic acid concentration of 1.5% by volume;
gradient elution was programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A; and
the flow rate was 10 mL/min, the column temperature was 30° C., and the detection wavelength was 280 nm.

A fraction at a retention time of 26.5-27.5 min was collected according to the liquid chromatogram. The fraction was subjected to vacuum evaporation and freeze-drying to obtain a first purified Melastoma dodecandrum proanthocyanidin analog. The first purified Melastoma dodecandrum proanthocyanidin analog was re-injected into the preparative liquid chromatography, and subjected to secondary purification according to the above gradient elution program, and a fraction at 26.8-27.2 min was collected. The fraction was subjected to vacuum evaporation and freeze-drying to obtain a second purified Melastoma dodecandrum proanthocyanidin analog.

FIG. 2 shows an HPLC chromatogram of the first purified Melastoma dodecandrum proanthocyanidin analog; FIG. 3 shows an HPLC chromatogram of the second purified Melastoma dodecandrum proanthocyanidin analog. It can be seen from FIG. 2 that: the first purified Melastoma dodecandrum proanthocyanidin analog could only obtain a mixture containing Melastoma dodecandrum proanthocyanidin analog, and the mixture further contains other polyphenols, in which a purity of a target fraction proanthocyanidin trimer is less than 40%. It can be seen from FIG. 3 that the purity of the target compound proanthocyanidin trimer is increased to more than 95% after secondary chromatographic purification.

Through structural characterization, the Melastoma dodecandrum proanthocyanidin analog was identified as proanthocyanidin trimer. The structural formula is as follows:

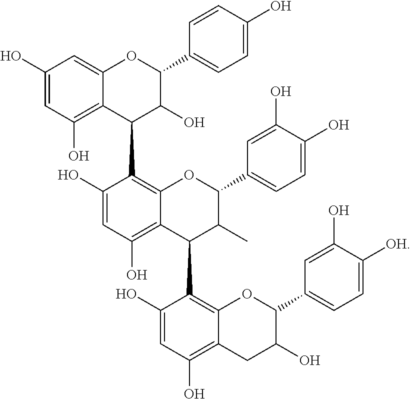

### Example 3 Evaluation of Inhibitory Activity of the Melastoma dodecandrum Proanthocyanidin Analog on DPP4

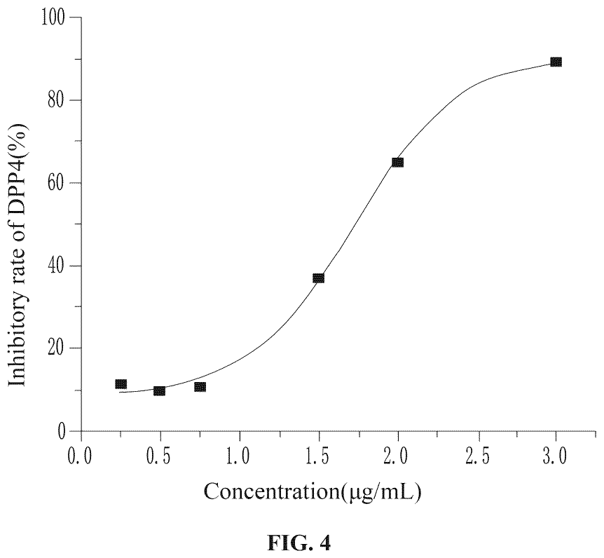

A series of concentrations of Melastoma dodecandrum proanthocyanidin analog solutions were prepared. 10 μL of a DPP4 solution (80 ng/mL) was dissolved in 140 μL of a Tris-HCl buffer (100 mM Tris-HCl, pH 7.8, consisting of 100 mM NaCl and 1 mM EDTA), and then 10 μL of each of the Melastoma dodecandrum proanthocyanidin analog solutions or positive control sitagliptin was added thereto to obtain systems. After incubation at room temperature for 10 min, 40 μL of substrate GP-AMC (0.1 mg/mL) was added to each of the systems. After mixing well, the fluorescence intensity emitted by the dissociation of the AMC group was continuously measured by a microplate reader for 15 min (excitation wavelength 360 nm; emission wavelength 460 nm). The result is shown in FIG. 4. FIG. 4 is a diagram showing the inhibitory effect of the Melastoma dodecandrum proanthocyanidin analog on DPP4. It can be seen from FIG. 4 that the Melastoma dodecandrum proanthocyanidin analog could effectively inhibit the activity of the DPP4, and median inhibitory concentration (IC₅₀) of the Melastoma dodecandrum proanthocyanidin analog is 1.73 g/mL. Therefore, the Melastoma dodecandrum proanthocyanidin analog has strong inhibitory activity against DPP4.

### Example 4 Evaluation of the Melastoma dodecandrum Proanthocyanidin Analog in Reducing Postprandial Blood Glucose (PBG)

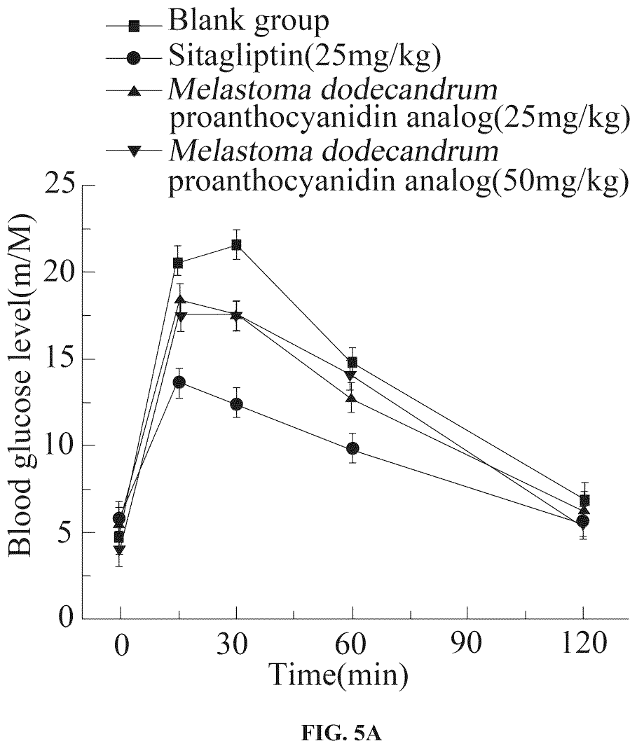

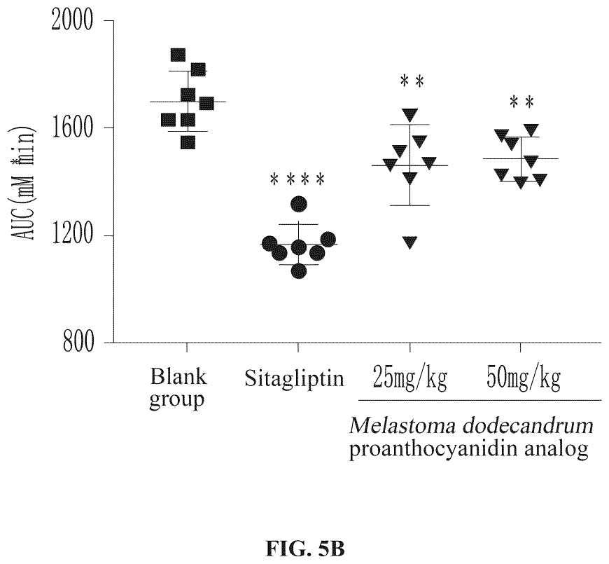

Thirty-two ICR mice (8-week-old) were randomly divided into four groups of eight mice. A control group was given normal saline; a positive control group was treated with sitagliptin at a dose of 25 mg/kg body weight (BW); a low-dose Melastoma dodecandrum proanthocyanidin analog group was administered at a dose of 25 mg/kg BW, while a high-dose Melastoma dodecandrum proanthocyanidin analog group was administered at a dose of 50 mg/kg BW. All mice were acclimatized for two weeks, followed by official experiment. Glucose was administered by gastric gavage 5 min after administration, and the PBG levels of mice were measured by tail-cutting at 0 min, 15 min, 30 min, 60 min, 90 min, and 120 min, respectively. The results are shown in FIGS. 5A-5B. FIGS. 5A-5B are diagrams showing the hypoglycemic effect of Melastoma dodecandrum proanthocyanidin analog in animals, where FIG. 5A illustrates the changes in PBG over time after gastric gavage of the glucose, and FIG. 5B illustrates the area under the concentration-time curve (AUC). It can be seen from FIGS. 5A-5B that the PBG level of mice increases rapidly after gastric gavage of the glucose, and peaks at 30 min, with a blood glucose concentration of 21.6±1.7 mM. After that, the PBG level begins to decrease gradually. At 2 h postprandially, its blood glucose level decreases to 6.9±0.9 mM, which is basically restored to the preprandial level (4.9±0.9 mM). Compared with the control group, treatment with 25 and 50 mg/kg BW of Melastoma dodecandrum proanthocyanidin analog (PAT) could significantly reduce PBG levels at 15 and 30 min postprandially. By calculating the AUC, it is found that treatment with 25 and 50 mg/kg BW of Melastoma dodecandrum proanthocyanidin analog (PAT) could reduce the AUC values of 2 h postprandial blood glucose by 14.1% and 12.6%, respectively, indicating that the Melastoma dodecandrum proanthocyanidin analog has an effect of reducing PBG.

### Comparative Example 1

The preparation process is the same as that of Example 2, except that the fraction collection time during preparative liquid chromatography purification was changed. If the fraction collection time is not in a range of 26.5-27.5 min, the fraction containing Melastoma dodecandrum proanthocyanidin analog cannot be obtained. If the fraction collection time is included and wider than the range of 26.5-27.5 min, the purity of the finally isolated Melastoma dodecandrum proanthocyanidin analog will be affected.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A method for extracting a Melastoma dodecandrum proanthocyanidin analog, comprising the following steps:
subjecting Melastoma dodecandrum fruit to alcohol extraction to obtain a crude extract of Melastoma dodecandrum Lour.;
subjecting the crude extract of the Melastoma dodecandrum Lour to extraction with ethyl acetate to obtain a Melastoma dodecandrum extract; and
subjecting the Melastoma dodecandrum extract to preparative liquid chromatography purification to obtain the Melastoma dodecandrum proanthocyanidin analog, wherein
the preparative liquid chromatography purification is conducted under parameters comprising:
a mobile phase A is pure acetonitrile, and a mobile phase B is an aqueous formic acid solution with a formic acid concentration of 1% to 2% by volume;
gradient elution is programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A;
a flow rate is 8-10 mL/min, a column temperature is 30° C., and a detection wavelength is 280 nm; and
a fraction at a retention time of 26.5-27.2 min is collected based on a liquid chromatogram.

2. The method of claim 1, wherein a reagent for the alcohol extraction is an ethanol solution; the ethanol solution has a volume concentration of 50% to 80%; the ethanol solution further comprises a 0.1% by volume of acid; and the acid is one selected from the group consisting of hydrochloric acid, formic acid, and acetic acid.

3. The method of claim 1, wherein the alcohol extraction is conducted at a feed-to-liquid ratio of 1 g:4 mL to 1 g:20 mL;
the alcohol extraction is performed by one selected from the group consisting of magnetic stirring extraction in a water bath and ultrasonic extraction; and
the alcohol extraction is conducted for 60-240 min.

4. The method of claim 1, wherein after the alcohol extraction, the method further comprises: subjecting a resulting extraction material to vacuum filtration to obtain a filtrate; and subjecting the filtrate to evaporation to remove a reagent for the alcohol extraction from the filtrate,
wherein the evaporation is rotary vacuum evaporation, and the rotary vacuum evaporation is conducted at a temperature of 40° C. to 60° C.

5. The method of claim 1, wherein a volume ratio of the ethyl acetate to the crude extract of the Melastoma dodecandrum Lour. is in a range of 0.5:1 to 3:1, and the extraction is conducted 2-6 times.

6. The method of claim 1, wherein after the extraction, an ethyl acetate phase is collected; the ethyl acetate phase is subjected to evaporation to remove the ethyl acetate from the ethyl acetate phase; the evaporation is rotary vacuum evaporation, and the rotary vacuum evaporation is conducted at a temperature of 40° C. to 50° C.

7. The method of claim 1, wherein the preparative liquid chromatography purification is conducted twice; and a second preparative liquid chromatography purification is conducted under parameters comprising:
the mobile phase A is the pure acetonitrile, and the mobile phase B is the aqueous formic acid solution with the formic acid concentration of 1% to 2% by volume;
the gradient elution is programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A;
the flow rate is 8-10 mL/min, the column temperature is 30° C., and the detection wavelength is 280 nm; and
the fraction at a retention time of 26.8-27.2 min is collected based on the liquid chromatogram.

8. The method of claim 1, wherein after the fraction is collected, the method further comprises subjecting the fraction to vacuum evaporation and freeze-drying in sequence.

9. A Melastoma dodecandrum proanthocyanidin analog prepared by the method of claim 1, wherein an active pharmaceutical ingredient of the Melastoma dodecandrum proanthocyanidin analog is a proanthocyanidin trimer.

10. A hypoglycemic method, comprising administrating the Melastoma dodecandrum proanthocyanidin analog of claim 9 in a subject in need thereof.

11. The method of claim 2, wherein the alcohol extraction is conducted at a feed-to-liquid ratio of 1 g:4 mL to 1 g:20 mL;
the alcohol extraction is performed by one selected from the group consisting of magnetic stirring extraction in a water bath and ultrasonic extraction; and
the alcohol extraction is conducted for 60-240 min.

12. The method of claim 2, wherein after the alcohol extraction, the method further comprises: subjecting a resulting extraction material to vacuum filtration to obtain a filtrate; and subjecting the filtrate to evaporation to remove the reagent for the alcohol extraction from the filtrate,
wherein the evaporation is rotary vacuum evaporation, and the rotary vacuum evaporation is conducted at a temperature of 40° C. to 60° C.

13. The method of claim 5, wherein after the extraction, an ethyl acetate phase is collected; the ethyl acetate phase is subjected to evaporation to remove the ethyl acetate from the ethyl acetate phase; the evaporation is rotary vacuum evaporation, and the rotary vacuum evaporation is conducted at a temperature of 40° C. to 50° C.

14. The method of claim 7, wherein after the fraction is collected, the method further comprises subjecting the fraction to vacuum evaporation and freeze-drying in sequence.

15. The Melastoma dodecandrum proanthocyanidin analog of claim 9, wherein a reagent for the alcohol extraction is an ethanol solution; the ethanol solution has a volume concentration of 50% to 80%; the ethanol solution further comprises a 0.1% by volume of acid; and the acid is one selected from the group consisting of hydrochloric acid, formic acid, and acetic acid.

16. The Melastoma dodecandrum proanthocyanidin analog of claim 9, wherein the alcohol extraction is conducted at a feed-to-liquid ratio of 1 g:4 mL to 1 g:20 mL;
the alcohol extraction is performed by one selected from the group consisting of magnetic stirring extraction in a water bath and ultrasonic extraction; and
the alcohol extraction is conducted for 60-240 min.

17. The Melastoma dodecandrum proanthocyanidin analog of claim 9, wherein after the alcohol extraction, the method further comprises: subjecting a resulting extraction material to vacuum filtration to obtain a filtrate; and subjecting the filtrate to evaporation to remove a reagent for the alcohol extraction from the filtrate,
wherein the evaporation is rotary vacuum evaporation, and the rotary vacuum evaporation is conducted at a temperature of 40° C. to 60° C.

18. The Melastoma dodecandrum proanthocyanidin analog of claim 9, wherein a volume ratio of the ethyl acetate to the crude extract of the Melastoma dodecandrum Lour. is in a range of 0.5:1 to 3:1, and the extraction is conducted 2-6 times.

19. The Melastoma dodecandrum proanthocyanidin analog of claim 9, wherein after the extraction, an ethyl acetate phase is collected; the ethyl acetate phase is subjected to evaporation to remove the ethyl acetate from the ethyl acetate phase; the evaporation is rotary vacuum evaporation, and the rotary vacuum evaporation is conducted at a temperature of 40° C. to 50° C.

20. The Melastoma dodecandrum proanthocyanidin analog of claim 9, wherein the preparative liquid chromatography purification is conducted twice; and a second preparative liquid chromatography purification is conducted under parameters comprising:
the mobile phase A is the pure acetonitrile, and the mobile phase B is the aqueous formic acid solution with the formic acid concentration of 1% to 2% by volume;
the gradient elution is programmed as follows: at 0-4 min, 5-20% of the mobile phase A; at 4-18 min, 20-25% of the mobile phase A; at 18-21 min, 25-35% of the mobile phase A; at 21-24 min, 35-60% of the mobile phase A; at 24-27 min, 60-5% of the mobile phase A; and at 27-30 min, 5% of the mobile phase A;
the flow rate is 8-10 mL/min, the column temperature is 30° C., and the detection wavelength is 280 nm; and
the fraction at a retention time of 26.8-27.2 min is collected based on the liquid chromatogram.
